(19) United States
(12) Patent Application Publication
(10) Pub. No.: US 2008/0058893 A1
(43) Pub. Date: Mar. 6, 2008

(54) Deep Brain Stimulation System

(75) Inventors: Elke Naujokat, Aachen (DE); Josef Lauter, Geilenkirchen (DE); Matthew Harris, Aachen (DE); Guido Josef Muesch, Linnich (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V, Eindhoven (NL)

(21) Appl. No.: 11/575,323

(22) PCT Filed: Sep. 9, 2005
(86) PCT No.: PCT/IB05/52949

§ 371 Date: Mar. 15, 2007

(30) Foreign Application Priority Data
Sep. 20, 2004 (EP) 04104535.2

(51) Int. Cl. A61N 1/05

(52) U.S. Cl. 607/45

(57) ABSTRACT

The invention provides automatic control of a deep brain stimulation system (2) using tremor detection based on accelerometer signals. Because no manual interventions are necessary, the ease of use for the patient (1) is greatly enhanced. Furthermore the autonomy of the patients in normal daily life is increased. The present invention also results in a reduced energy consumption and consequently longer battery life. On the other side with the present invention an optimal treatment is possible, adjusted to the patient's symptoms without the help of a physician.

The present invention relates to a deep brain stimulation system. Furthermore the invention relates to a method of controlling a generator adapted to generate electrical signals for deep brain stimulation and to a computer program for carrying out said method.

Parkinson's disease is one of the most frequent neurological diseases. This disease affects approximately 2 out of 1000 people and is associated with damage to a part of the brain that controls muscle movement. Usually the first symptom of Parkinson's disease is tremor (trembling or shaking) of a limb, especially when the body is at rest. The tremor often begins on one side of the body, frequently in one hand. The most common treatment of Parkinson's disease is medication with L-dopa. Thus the cardinal symptoms tremor, bradykinesia, rigor and posture instability can be alleviated effectively during the first years of the disease. After five to ten years, complications such as relative loss of efficiency or strong effect fluctuations occur increasingly, leading to considerable impairment of the patients' quality of life. For those Parkinson patients, who are seriously handicapped by their tremor despite optimal medication, neurosurgical procedures or the implantation of a deep brain stimulation system are used. By electrical stimulation the affected brain regions can be “blocked”, so that the symptoms are “switched off”. The treatment with a deep brain stimulation system does not stop the illness, but alleviates the symptoms. Thus the patient's quality of life improves clearly.

However, the handling of known deep brain stimulation systems is rather complex. Known systems are adapted to be programmed by physicians in order to adjust the electrical stimulation to the patient's situation, e.g. to the severity of the symptoms. For such an adjustment the patient has to contact the physician from time to time. Thus a permanent optimal treatment is not possible.

Furthermore the systems require the patients to manually turn the system on and off, e.g. at night time to conserve battery power. For this purpose a magnet has to be moved by the patient to a specific part of the system. This presents considerable difficulty to many patients whose tremor significantly impairs arm functions, as they are unable to hold a magnet in a stable manner. Consequently, many patients are unable to turn their stimulation systems on or off without assistance.

In the international patent application WO 85/01213 a neurocybernetic prosthesis is disclosed, wherein a pulsed electrical signal is applied to the vagus nerve in order to control or prevent involuntary movements. Thereby EEG signals are used to activate the prosthesis. Disadvantages of this technique are the weak EEG signals and the large influence of noise.

Demand-controlled deep brain stimulation techniques for the therapy of movement disorders like severe Parkinson's disease or essential tremor are known from “Obsessive-Compulse Disorder: Development of Demand-Controlled Deep Brain Stimulation with methods from Stochastic Phase resetting” in Neuropsychopharmacology (2003) 28, S27-S34. These techniques are again based on the use of EEG signals and therefore show the same disadvantages as described above.

It is an object of the present invention to improve the handling of a system for stimulating the nervous system of a patient.

This object is achieved according to the invention by a system for deep brain stimulation, the system comprising a generator adapted to generate electrical signals, an electrode adapted to stimulate the brain depending on the generated signals and a sensor adapted to sense tremor, i.e. an involuntary, often rhythmic and oscillating movement of any body part, mainly caused by contractions of reciprocally innervated antagonist muscles. Furthermore the system comprises a controller adapted to control the generator depending on sensor data. All appliances of the system are adapted in a way to form a deep brain stimulation system. A pulse generator is used to generate electric impulses which are applied to the patient by the actuator. The electrode is preferably implanted into the brain of the patient in order to stimulate certain parts of the brain.

The object of the present invention is also achieved by a method of controlling a generator adapted to generate electrical signals for deep brain stimulation, the method comprising the steps of sensing tremor and controlling the generator depending on sensor data.

The object of the present invention is also achieved by a computer program comprising computer instructions adapted to control the generator depending on sensor data of said sensor when the computer program is executed in a computer. The technical effects necessary according to the invention can thus be realized on the basis of the instructions of the computer program in accordance with the invention. Such a computer program can be stored on a carrier or it can be available over the internet or another computer network. Prior to executing the computer program is loaded into the computer by reading the computer program from the carrier, for example by means of a CD-ROM player, or from the internet, and storing it in the memory of the computer. The computer includes inter alia a central processor unit (CPU), a bus system, memory means, e.g. RAM or ROM etc. and input/output units.

The present invention enables a user-friendly stimulation system. Because no manual interventions are necessary, the ease of use for the patient is greatly enhanced. Furthermore the autonomy of the patients in normal daily life is increased. The present invention also results in a reduced energy consumption and consequently longer battery life. On the other side with the present invention an optimal treatment is possible, adjusted to the patient's symptoms without the help of a physician.

The present invention suggests to implement a closed-loop system, wherein a feedback from the output is used to control the input. In other words the treatment of the patient influences the patient's body functions and the body functions are the basis for any further treatment.

These and other aspects of the invention will be further elaborated on the basis of the following embodiments which are defined in the dependent claims.

For sensing tremor preferably accelerometers are used as sensors. The use of accelerometers is especially advantageous, because they are small, easy to use, available with one to three sensing axes and cheap. They can be easily integrated into small and convenient patient devices or even integrated into his clothing. Any kind of accelerometer might be used, such as pendulous accelerometers, vibrational accelerometers or electromagnetic accelerometers. The sensors can be realized as wrist-worn or ankle-worn devices. Alternatively the sensors can be integrated into the clothing of the patient, e.g. long sleeves of shirts or socks. Another alternative is to implant the sensors, e.g. under the skin of the patient.

The data sensed by the sensors are preferably transmitted to the controller by means of a wireless communication link, using e.g. radio transmission, Bluetooth or another technique. For this purpose at least one communication unit is provided. Preferably each sensor comprises its own communication unit, i.e. its own transmitter to send its data to the controller.

The generator is preferably controlled by the controller depending on sensor data. In an embodiment of the invention the controller is adapted to turning the generator on and/or off depending on these data. For example the controller is adapted to switch on the stimulation system for an adjustable period of time, e.g. 30 minutes, if the sensor detects tremor. A manual intervention of the patient is not necessary.

In another embodiment the controller is adapted to control the form or the nature of the electrical signals generated by the generator depending on the sensor data. In other words the controller controls the generator to perform an automatic adjustment of voltage amplitude, pulse width and/or impulse frequency. to the severity of; the actual tremor. The treatment can be adjusted automatically without assistance of a physician or another person.

The controller preferably comprises a data processing unit to process the received sensor data. The data processing unit can be realized as a hardware data processor or as a computer program designed for carrying out data processing or a combination of both. Depending on the function to be realized by the controller, the data processing unit either simply detects a certain body function, e.g. tremor in contrast to normal movements of the patient, or computes a stimulation treatment to be applied to the patient depending on the severity of the tremor.

In a further embodiment of the invention the controller is part of the generator. In other words the generator contains a controller unit adapted to receive sensor data and further adapted to control the generator accordingly. Generator and controller are preferably implanted into the patient's body, e.g. under the skin of the chest. Alternatively the controller is provided outside the generator, e.g. as part of one of the sensors. In this case the controller is adapted to establish a communication link to the generator. If existing generators already comprise an input unit to receive such control signals, they can be used with the present invention.

These and other aspects of the invention will be described in detail hereinafter, by way of example, with reference to the following embodiments and the accompanying drawings, in which:

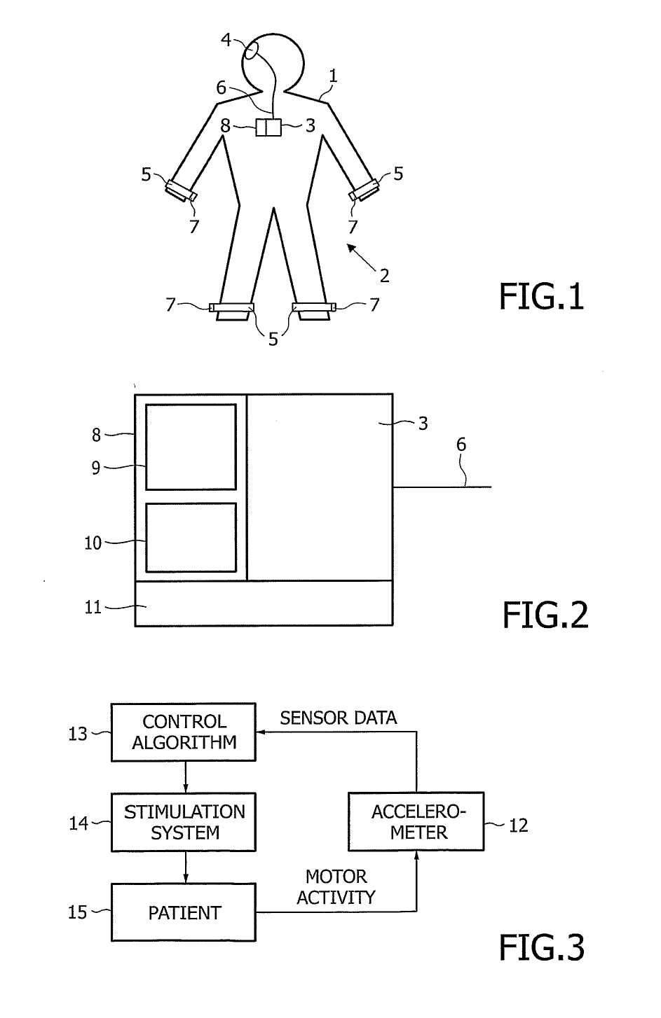

FIG. 1 is a schematic picture of a patient using the system according to the invention;

FIG. 2 is a block diagram showing the closed-loop system according to the invention;

FIG. 3 is a block diagram showing the pulse generator according to the invention.

FIG. 1 illustrates a patient 1 suffering from Parkinson's disease. A deep brain stimulation system 2 is used to treat the patient 1. The system 2 basically comprises a pulse generator 3, an electrode 4 and one or several sensor units 5. The pulse generator 3 is adapted to generate electrical pulses having a pulse frequency of between 2 and 250 cycles per second, a pulse duration of between 60 and 450 microseconds and a voltage amplitude of between 0 and 10.5 Volts. For a standard tissue impedance in the range of 1000 Ohms a current amplitude of up to 10 milliamperes therefore falls within the therapeutic range of deep brain stimulation. The pulse generator 3 is implanted like a cardiac pacemaker into the patient's chest and the electrode 4 is implanted in those regions of the patient's brain which are affected by the disease. For the electrode 4 preferably a material that is inert to chemical reactions with the surrounding tissue, e.g. titanium, platinum, gold or an alloy containing these or comparable materials is used. Furthermore the electrodes must be mechanically stable and biologically compatible. As one embodiment of the sensing apparatus micro-wire electrodes with the ability to record from individual neurons or small populations of neurons can be used. Alternatively, synthetic electrical-biological interfaces in which metal and silicone electrical substrates are coupled with biological substrates such as nerve growth factors can be used. Generator 3 and electrode 4 are connected to each other by electrode leads 6 pass from the generator 3 to the brain through a subcutaneous tunnel.

Electromagnetic accelerometers are used as sensor units 5 to sense the motor activity of the patient 1. The sensor units 5 are mounted onto the patient's wrists and ankles. The sensor units 5 are adapted to sense the motor activity of the patient 1 continuously, e.g. at a frequency of 20 Hz (or even higher). Each sensor unit 5 comprises a communication unit 7 to establish a radio communication link to a controller 8. The sensor units 5 are adapted to send the sensor data to the controller 8 in frequent intervals. Preferably sensor data are sent immediately after the sensing has finished, i.e. they are sent continuously to the controller.

The controller 8 is an integrated part of the generator 3 such that there is a direct link between the controller 8 and the generator 3, as depicted in FIG. 3. In order to process the received sensor data the controller 8 comprises a data processing unit 9 (computer) employing a signal processor. The signal processor analyses sensor data received from the sensor units 8 according to a defined analyzing algorithm.

In a first embodiment the sensor data are used to analyze the motor activity of the patient 1 in order to detect tremor. For example a tremor detect signal is generated every time the movements of the patient's hand or foot are rhythmic and oscillating in a certain predetermined way. According to the control algorithm of the controller 8, the generator 3 is turned on, if a tremor detect signal is generated. In other words the deep brain stimulation system 2 is activated depending on the state of the patient 1. Preferably the generator 3 is turned off automatically after a predetermined period of time, e.g. after 30 minutes.

In another embodiment not only the presence of tremor but the intensity of tremor is analyzed. If the intensity exceeds a predetermined level or a certain tremor characteristic is registered over a predetermined period of time, the control algorithm carried out in the data processing unit 9 automatically changes the nature of the generated electrical signals, e.g. adjusts the impulse frequency, amplitude or pulse duration etc. according to medical requirements. These automatic modifications are also carried out if tremor is detected despite stimulation. Every patient receives his very own treatment, perfectly adjusted to his individual medical requirements and needs. For this purpose the control algorithm used is adapted to provide flexible changes of treatment parameters.

In still another embodiment of the invention the deep brain stimulation system 2 is adapted to determine time periods in which the patient 1 sleeps. For this purpose an additional sensor unit 5 is mounted onto the patient's torso to provide (at least two-dimensional) sensor data for determining the position of the patient. Furthermore an electrocardiogram is attached to the patient's torso to monitor the patient's heart rate, which decreases during sleep. If the patient is in a horizontal position with a heart rate falling below a certain value the deep brain stimulation is turned off automatically.

Analyzing and control algorithms are provided to the controller 8 prior to implantation. Preferably both algorithms can be updated by transferring algorithms to the data processing unit 9. The controller 8 is adapted to allow external access, i.e. via an integrated input module 10 for wireless communication. The same input module 10 is used for receiving sensor data from the sensor units 5. A battery 11 provides energy to the controller 8 and the generator 3.

The present invention suggests to implement a closed-loop system, wherein a feedback from the output is used to control the input, see FIG. 2. In other words the treatment of the patient 1 influences the patient's body functions and the body functions are the basis for any further treatment. If the measuring system 12 (accelerometers) detects tremor, the control system 13 by means of the control algorithm activates the actuator 14 (deep brain stimulation system) in order to treat the controlled system 15 (the Parkinson patient). Depending on the motor activity of the controlled system 15 the measuring system 12 again receives data which subsequently are used to control the actuator 14.

The technique of a closed-loop system is used in order to treat the patient 1 in a best possible manner. The stimulation is activated only in case the patient 1 needs treatment. Furthermore the treatment can be adapted to the tremor situation of the patient 1. If, for example, the controller adjusts the impulse frequency depending on the severity of a tremor attack in a first step and the feedback (motor activity) given by the patient 1 is not satisfying according to a medical point of view, the control algorithm can automatically adjust in one or more subsequent steps the impulse frequency even further until the motor activity of the patient 1 corresponds to a satisfying or normal level.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” does not exclude a plurality, and that a single element, such as a computer system or another unit may fulfil the functions of several means recited in the claims. Any reference signs in the claims shall not be construed as limiting the claim concerned.

### REFERENCE LIST

1 patient
2 deep brain stimulation system
3 pulse generator
4 electrode
5 sensor
6 lead
7 communication unit
8 controller
9 data processing unit
10 input module
11 battery
12 measuring system
13 control system
14 actuator
15 controlled system

1. A deep brain stimulation system (2), the system (2) comprising
a generator (3) adapted to generate electrical signals,
an electrode (4) adapted to stimulate the brain depending on the generated signals,
a sensor (5) adapted to sense tremor and
a controller (8) adapted to control the generator (3) depending on sensor data.

2. The system (2) as claimed in claim 1, wherein an accelerometer is used as sensor (5).

3. The system (2) as claimed in claim 1, wherein the controller (8) is adapted to turning the generator (3) on and/or off depending on sensor data.

4. The system (2) as claimed in claim 1, wherein the controller (8) is adapted to control the form of the electrical signals generated by the generator (3) depending on sensor data.

5. The system (2) as claimed in claim 1, wherein the controller (8) is part of the generator (3).

6. A method of controlling a generator (3) adapted to generate electrical signals for a deep brain stimulation electrode (4), the method comprising the steps:
sensing tremor by a sensor (5),
controlling the generator (3) depending on sensor data by a controller (8).

7. A computer program for controlling a generator (3) adapted to generate electrical signals for a deep brain stimulation electrode (4), comprising
computer instructions to control the generator (3) depending on sensor data of a sensor (5) adapted to sense tremor.
